SEQUENCE LISTING (12 sequences)

SEQ ID NO: 1 - DNA, 1974 bp, Staphylococcus aureus
atggctaaagaaacattttatataacaaccccaatatactatcctagtgggaatttacatataggacatgcatattctacagtggctggagatgttattgcaagatataagagaatgcaaggatatgatgttcgttatttgactggaacggatgaacacggtcaaaaaattcaagaaaaagctcaaaaagctggtaagacagaaattgaatatttggatgagatgattgctggaattaaacaattgtgggctaagcttgaaatttcaaatgatgattttatcagaacaactgaagaacgtcataaacatgtcgttgagcaagtgtttgaacgtttattaaagcaaggtgatatctatttaggtgaatatgaaggttggtattctgttccggatgaaacatactatacagagtcacaattagtagacccacaatacgaaaacggtaaaattattggtggcaaaagtccagattctggacacgaagttgaactagttaaagaagaaagttatttctttaatattagtaaatatacagaccgtttattagagttctatgaccaaaatccagattttatacaaccaccatcaagaaaaaatgaaatgattaacaacttcattaaaccaggacttgctgatttagctgtttctcgtacatcatttaactggggtgtccatgttccgtctaatccaaaacatgttgtttatgtttggattgatgcgttagttaactatatttcagcattaggctatttatcagatgatgagtcactatttaacaaatactggccagcagatattcatttaatggctaaggaaattgtgcgattccactcaattatttggcctattttattgatggcattagacttaccgttacctaaaaaagtctttgcacatggttggattttgatgaaagatggaaaaatgagtaaatctaaaggtaatgttgtagaccctaatattttaattgatcgctatggtttagatgctacacgttattatctaatgcgtgaattaccatttggttcagatggcgtatttacacctgaagcatttgttgagcgtacaaatttcgatctagcaaatgacttaggtaacttagtaaaccgtacgatttctatggttaataagtactttgatggcgaattaccagcgtatcaaggtccacttcatgaattagatgaagaaatggaagctatggctttagaaacagtgaaaagctacactgaaagcatggaaagtttgcaattttctgtggcattatctacggtatggaagtttataagtagaacgaataagtatattgacgaaacaacgccttgggtattagctaaggacgatagccaaaaagatatgttaggcaatgtaatggctcacttagttgaaaatattcgttatgcagctgtattattacgtccattcttaacacatgcgccgaaagagatttttgaacaattgaacataaacaatcctcaatttatggaatttagtagtttagagcaatatggtgtgcttactgagtcaattatggttactgggcaacctaaacctattttcccaagattggatagcgaagcggaaattgcatatatcaaagaatcaatgcaaccgcctgctactgaagaggaaaaagaagagattcctagcaaacctcaaattgatattaaagactttgataaagttgaaattaaggcagcaacgattattgatgctgaacatgttaagaagtcagataagcttttaaaaattcaagtagacttagattctgaacaaagacaaattgtatcaggaattgccaaattctatacaccagatgatattattggtaaaaaagtagcagttgttactaacctgaaaccagctaaattaatgggacaaaaatctgaaggtatgatattatctgctgaaaaagatggtgtattaaccttagtaagtttaccaagtgcaattccaaatggtgcagtgattaaataa

SEQ ID NO: 2 - DNA, 1974 bp, Staphylococcus aureus; identical to SEQ ID NO: 1
  CDS (1)..(1974)

SEQ ID NO: 3 - protein, 657 aa, Staphylococcus aureus
MAKETFYITTPIYYPSGNLHIGHAYSTVAGDVIARYKRMQGYDVRYLTGTDEHGQKIQEKAQKAGKTEIEYLDEMIAGIKQLWAKLEISNDDFIRTTEERHKHVVEQVFERLLKQGDIYLGEYEGWYSVPDETYYTESQLVDPQYENGKIIGGKSPDSGHEVELVKEESYFFNISKYTDRLLEFYDQNPDFIQPPSRKNEMINNFIKPGLADLAVSRTSFNWGVHVPSNPKHVVYVWIDALVNYISALGYLSDDESLFNKYWPADIHLMAKEIVRFHSIIWPILLMALDLPLPKKVFAHGWILMKDGKMSKSKGNVVDPNILIDRYGLDATRYYLMRELPFGSDGVFTPEAFVERTNFDLANDLGNLVNRTISMVNKYFDGELPAYQGPLHELDEEMEAMALETVKSYTESMESLQFSVALSTVWKFISRTNKYIDETTPWVLAKDDSQKDMLGNVMAHLVENIRYAAVLLRPFLTHAPKEIFEQLNINNPQFMEFSSLEQYGVLTESIMVTGQPKPIFPRLDSEAEIAYIKESMQPPATEEEKEEIPSKPQIDIKDFDKVEIKAATIIDAEHVKKSDKLLKIQVDLDSEQRQIVSGIAKFYTPDDIIGKKVAVVTNLKPAKLMGQKSEGMILSAEKDGVLTLVSLPSAIPNGAVIK

SEQ ID NO: 4 - DNA, 19 bp, Artificial
  primer
acattacgaggaggaacag

SEQ ID NO: 5 - DNA, 19 bp, Artificial
  primer
ggtgtaaatacgccatctg

SEQ ID NO: 6 - DNA, 19 bp, Artificial
  primer
gtctttgcacatggttgga

SEQ ID NO: 7 - DNA, 19 bp, Artificial
  primer
tgcttctctagcacgtgta

SEQ ID NO: 8 - DNA, 33 bp, Artificial
  primer
caccatggctaaagaaacattttatataacaac

SEQ ID NO: 9 - DNA, 35 bp, Artificial
  primer
gactgtcgactattatttaatcactgcaccatttg

SEQ ID NO: 10 - DNA, 19 bp, Artificial
  primer
ggtgtaaatacgccatctg

SEQ ID NO: 11 - DNA, 20 bp, Artificial
  primer
tagttattgctcagcggtgg

SEQ ID NO: 12 - DNA, 20 bp, Artificial
  primer
taatacgactcactataggg

(19) United States
(12) Patent Application Publication
(10) Pub. No.: US 2008/0138835 A1
(43) Pub. Date: Jun. 12, 2008

(54) Mutants of Staphylococcal Mrs and Methods of Use

(75) Inventors: Ian A. Critchley, Lafayette, CO (US); Urs Ochsner, Denver, CO (US); James M. Bullard, Longmont, CO (US)

Correspondence Address: SWANSON & BRATSCHUN, L.L.C., 8210 SOUTHPARK TERRACE, LITTLETON, CO, 80120, US

(73) Assignee: REPLIDYNE, INC., Louisville, CO (US)

(21) Appl. No.: 11/568,765

(22) PCT Filed: May 9, 2005
(86) PCT No.: PCT/US2005/016379

§ 371 Date: Apr. 5, 2007

Related U.S. Application Data
(60) Provisional application No. 60/568,888, filed on May 7, 2004.

(51) Int. Cl. G01N 33/573; G01N 33/566; C07K 14/00

(52) U.S. Cl. 435/7.4; 436/501; 530/350

(57) ABSTRACT

Mutant bacterial MRS nucleic acids and proteins are provided, as well as uses for the nucleic acids and proteins, such as in a high-throughput screen for inhibitors of the mutant proteins.

### FIELD OF THE INVENTION

The invention generally relates to nucleic acids and polypeptides encoded by the nucleic acids, as well as uses for the nucleic acids and polypeptides. The invention also concerns methods and materials for the discovery and characterization of mechanisms of drug resistance.

### BACKGROUND OF THE INVENTION

Methicillin resistant S. aureus (MRSA) is a major cause of nosocomial infections with severe morbidity and mortality worldwide. According to a recent surveillance study, the methicillin (oxacillin) resistance rates among S. aureus ICU isolates from the United States, Canada, and Europe ranged from 19.7% to 59.4%. In the U.S., 42.8% of all S. aureus isolates in 2003 were methicillin-resistant. In the UK, the MRSA rate has increased by 5% in 2003-2004 to reach 40%. MRSA outbreaks in the community are also on a sharp incline. This rising global health and socio-economic problem demands new measures for both prevention and control of MRSA. Efforts have been made to decrease nosocomial MRSA outbreaks through identification of MRSA colonization at hospital admission followed by adequate interventions. The elimination of nasal carriage could prevent infection, thus, decolonization of carriers, especially in high risk groups, seems an adequate goal and warrants the discovery and development of new remedies either by chemical modification of existing antibiotics or by developing synthetic molecules with novel mechanisms of action. Many different topical agents and remedies have the potential to eliminate MRSA from the anterior nares or from the skin of carriers and thereby decrease the risk of subsequent infection, including mupirocin, known as Bactroban ointment, fusidic acid, indolmycin, silver sulphadiazine with cerium nitrate, chlorhexidine, tea tree oil, garlic-derived allicin extract, and autolysins such as lysostaphin. Mupirocin, an inhibitor of isoleucyl tRNA synthetase (IRS), has been used for many years as a topical agent to eliminate nasal carriage of S. aureus and for the treatment of impetigo due to staphylococci and streptococci. However, resistance to mupirocin is on the rise and its effectiveness is compromised in areas where clinical use is high, leading to persistence or recolonization. According to the SENTRY antimicrobial surveillance program in 2000, 1.9-5.6% of S. aureus isolates and 12.8-39.9% of coagulase-negative staphylococci (CoNS) isolated in the United States, Canada, Latin America, and Europe were mupirocin-resistant. Both low-level mupirocin resistance (MIC=8-256 μg/ml) and high-level resistance (MIC>256 μg/ml) have been described. Low-level mupirocin resistance is caused by point mutations which have been mapped within the ileS gene encoding the isoleucyl tRNA synthetase 1. High-level mupirocin resistance is mediated through acquisition of a second isoleucyl tRNA synthetase gene, mupA.

Aminoacyl-tRNA synthetases carry out the condensation of a specific amino acid with its cognate tRNA species in a reaction that is dependent on ATP. Methionyl tRNA synthetase (MRS) represents a novel target that is essential and well conserved among Gram-positive microbes. Most bacteria, including S. aureus, contain a type I MRS, although a significant proportion of S. pneumoniae strains contain a second gene, metS2, which encodes a type 2 MRS that is more closely related to archeal synthetases. Both types of MRS belong to class I tRNA synthetases that harbor an ATP-binding Rossman-fold with the conserved H(M/I)GH and KMSKS motifs.

### SUMMARY OF THE INVENTION

The present invention provides mutant MRS polypeptides. In some embodiments, the mutant MRS polypeptide is a mutant S. aureus MRS polypeptide. In some embodiments the mutant S. aureus MRS polypeptide has at least one amino acid changed relative to SEQ ID NO:3 or a fragment of SEQ ID NO:3. In some embodiments, the mutant S. aureus MRS polypeptide is associated with decreased susceptibility to an antagonist of the polypeptide. In some embodiments, the mutant S. aureus MRS polypeptide is associated with decreased susceptibility to Compound 1. In some embodiments, the mutant S. aureus MRS polypeptide includes mutants with the following amino acid changes: L19F, T50A, E52D, G54S, G54A, I57N, I57T, Q58L, A61V, A61T, A64S, A64P, A77V, E98G, I94N, R100S, V108M, V108L, I238F, L213W, V215A, V215I, G223C, P230T, I238F, V242F, V242I, A247E, L257P, M269I, V296F, and combinations of the foregoing.

The present invention also provides methods for identifying ligands, inhibitors, and enhancers of MRS function. In one embodiment the method comprises, contacting a compound to be tested with an isolated mutant MRS polypeptide or polypeptide fragment; and determining whether the compound is a ligand of said polypeptide, wherein formation of a complex between the compound and the polypeptide is indicative that the compound is a ligand. In some embodiments, the method is performed in the presence of a natural ligand of the MRS polypeptide, such as methionine. The formation of a complex can by monitored by detecting or measuring direct binding, or synthetase activity or cellular response by said mutant MRS protein in response to binding of a ligand thereto.

The compound to be tested can be any compound, such as peptides, carbohydrates, vitamins, nucleic acids, botanically-derived compounds, organic compounds, pharmaceutical agents.

### BRIEF DESCRIPTION OF THE DRAWINGS

FIG. 1A shows the amino acid sequence for wild-type MRS from S. aureus. FIG. 1B shows the nucleotide acid sequence for wild-type MRS from S. aureus.

FIG. 2A shows the results of a macromolecular synthesis assay in S. pneumoniae R6 (A) and an isogenic spo rel mutant affected in stringent response (B). Cells were treated with Compound 1 for 10 min, and the incorporation of [5-³H]uridine and L-[4, 5-³H]leucine was determined to measure protein synthesis (▪) and RNA synthesis (▴).

FIG. 3 shows spontaneous resistance rates to Compound 1 in different S. aureus strains. Approximately 100 cells were plated on agar containing Compound 1 at concentrations ranging from 0.25-16 μg/ml and colonies were enumerated after 48 h.

FIGS. 4A and 4B show growth curves for S. aureus and isogenic metS mutants illustrating the fitness burden due to MRS mutations. The strains were grown in Mueller-Hinton broth in the absence of drug at 35° C. in flasks shaken at 200 rpm and the optical densities of the cultures were recorded.

FIG. 5 shows a model of S. aureus apo MRS. The E. coli MRS co-crystallized form (PDB ID=1F4L) is shown in FIG. 5A, with the methionine ligand shown in dark grey and labeled “Met”. FIG. 5B depicts a 3D model of S. aureus MRS apo form that was generated on the basis of the E. coli MRS apo form as a template (PDB ID=1QQT). The ten active site residues within MRS are shown in light grey, and the positions of the residues affected in S. aureus MRS mutants with decreased susceptibility to REP8839 are shown in black.

FIG. 6 shows a comparison of modeled active sites of S. aureus wild-type and mutant MRS. The key residues (labeled) proximal to the active site residues that outline the methionine/ATP binding pocket (grey) caused the largest shift in MIC for REP8839 when altered. Upper panel, wild-type MRS (157 G54) and mutant MRS(N57 S54). Lower panel, wild-type MRS (A61 A64) and mutant MRS (T61 S64).

FIG. 7 shows an SDS-PAGE analysis of total protein extracted from cell cultures expressing mutant S. aureus MRS proteins. The position and consequence of the mutation is shown at the bottom of gel. The lane labeled std is 10 kDa protein ladder (Gibco, Rockville, Md.). Induction of expression of S. aureus MRS by isopropyl β-D-thiogalactoside (IPTG) (+) and un-induced (−) are indicated at the top of the lanes.

### DETAILED DESCRIPTION OF THE INVENTION

This invention is directed to mutant MRS proteins and fragments thereof, vaccine and pharmaceutical compositions including mutant MRS proteins or fragments thereof, methods of preparing mutant MRS proteins and fragments thereof, and methods of using MRS proteins and fragments thereof. The present invention also relates to structural gene and amino acid sequences encoding mutant MRS proteins from a variety of bacteria. As used herein, the term “gene” refers to a nucleic acid (e.g., DNA) sequence that comprises coding sequences necessary for the production of a polypeptide or precursor (e.g., MRS, as appropriate). The polypeptide can be encoded by a full length coding sequence or by any portion of the coding sequence so long as the desired activity or functional properties (e.g., enzymatic activity, ligand binding, etc.) of the full-length or fragment are retained. The mRNA functions during translation to specify the sequence or order of amino acids in a nascent polypeptide.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited proteins. It is to be noted that the term “a” or “an” entity refers to one or more of that entity; for example, a protein refers to one or more proteins or at least one protein. As such, the terms “a” (or “an”), “one or more” and “at least one” can be used interchangeably herein. It is also to be noted that the terms “comprising”, “including”, and “having” can be used interchangeably. Furthermore, a compound “selected from the group consisting of” refers to one or more of the compounds in the list that follows, including mixtures (i.e., combinations) of two or more of the compounds.

A potent MRS type I inhibitor with antibacterial activity against staphylococci and enterococci has recently been identified as a high throughput screening hit. Subsequent medicinal chemistry efforts resulted in N-(4-bromo-5-(1-fluorovinyl)-3-methylthiophen-2-ylmethyl)-N′-(1H-quinolin-4-one)propane-1,3-diamine, (compound 1):

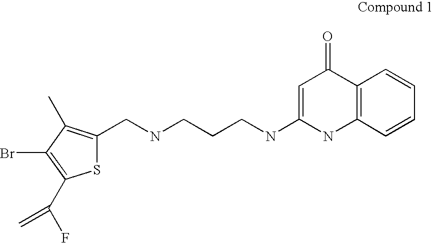

which is a fluorovinylthiophene-containing diaryldiamine with a promising antibacterial profile and spectrum of activity. Compound 1 is discussed in International Patent Application Publication No. WO 2005/009336A2, incorporated by reference herein in its entirety. The mode-of-action of Compound 1, including the characterization of laboratory-generated mutants with decreased susceptibility to this fully synthetic compound is disclosed herein. We have measured the inhibition of MRS activity by compound 1 and find that it is a highly potent (nanomolar) inhibitor of S. aureus MetS (Table 1). We noted that the IC50 for inhibition was as low as the concentration of enzyme present in the assay. Furthermore, the correlation between IC50 and enzyme concentration remained consistent as enzyme concentration was varied (data not shown). This behavior was noted previously for similar S. aureus MetS inhibitors and indicates that measurement of a true inhibitory constant is limited by the enzyme concentrations. At present it can be concluded at a minimum that the IC50 for Compound 1 inhibition of S. aureus MetS is <1.9 nM.

Target specificity of Compound 1 has also been demonstrated in whole-cell assays using S. aureus and in S. pneumoniae R6, as well as through extensive resistance studies. The spontaneous resistance rates for bacteriostatic agents such as Compound 1 or mupirocin were relatively high compared to published resistance rates for bactericidal drugs such as β-lactams or fluoroquinolones. Interestingly, Compound 1 was inferior to mupirocin regarding the resistance rates upon exposure to 2-16 μg/ml of drug. Compound 1 was essentially equal to mupirocin regarding the MPC (Mutant Prevention Concentration), and was superior to mupirocin regarding the level of resistance in stable spontaneous mutants. A large number of MRS mutations that reduced susceptibility to Compound 1 have been characterized and may prove useful to gain further insight in the drug-target interactions.

Mutant MRS proteins are proteins that have at least one amino acid change and optionally have at least one change in a biological function compared with a protein substantially corresponding to a wild type MRS protein. Mutant MRS proteins can be generated using a variety of methods including site-directed mutagenesis, random mutagenesis, conventional mutagenesis, in vitro mutagenesis, spontaneous mutagenesis, chemical synthesis, over-expression of the metS gene (encoding MRS) in the presence of ligands or inhibitors. Mutant MRS proteins are selected to ensure at least one change in an amino acid; and in some embodiments, to have a change in at least one biological function of the molecule. The mutant proteins are useful in vaccine compositions for protection against at least one biological activity of MRS such as prevention or amelioration of bacterial infection or in methods of treating humans or animals with symptoms of bacterial infection.

As used herein, a mutant MRS polypeptide, in one embodiment, is a polypeptide that is related to (i.e., bears structural similarity to) the S. aureus MRS polypeptide of about 657 amino acids and having the sequence depicted SEQ ID NO: 3. The original identification of such a polypeptide is detailed in Kuroda, et al., Lancet 357 (9264), 1225-1240 (2'001), and has accession number GenBank BAB41678 (ORFID:SA0448). The 1,974-bp metS gene which encodes the 75 kDa MRS appears to be very well conserved among different S. aureus strains. Analysis of MRS amino acid sequences obtained from 18 S. aureus strains (16 clinical isolates and 2 quality control strains) revealed one single variation. The residue at position 260 was arginine in 12 strains and lysine in 6 strains. In some embodiments, a mutant MRS polypeptide is encoded by a polynucleotide that hybridizes under stringent hybridization conditions to a gene encoding an S. aureus MRS polypeptide (i.e., an S. aureus gene).

It is to be noted that the term “a” or “an” entity refers to one or more of that entity; for example, a gene refers to one or more genes or at least one gene. As such, the terms “a” (or “an”), “one or more” and “at least one” can be used interchangeably herein. It is also to be noted that the terms “comprising,” “including,” and “having” can be used interchangeably.

As used herein, stringent hybridization conditions refer to standard hybridization conditions under which polynucleotides, including oligonucleotides, are used to identify molecules having similar nucleic acid sequences. Such standard conditions are disclosed, for example, in Sambrook et al., MOLECULAR CLONING: A LABORATORY MANUAL, Cold Spring Harbor Labs Press, 1989. Examples of such conditions are provided in the Examples section of the present application.

As used herein, an S. aureus gene includes all nucleic acid sequences related to a natural S. aureus MRS gene such as regulatory regions that control production of the S. aureus MRS polypeptide encoded by that gene (such as, but not limited to, transcription, translation or post-translation control regions) as well as the coding region itself. In one embodiment, an S. aureus MRS gene includes the nucleic acid sequence SEQ ID NO:1 having at least one base change. Nucleic acid sequence SEQ ID NO:1 represents the deduced sequence of a cDNA (complementary DNA) polynucleotide. It should be noted that since nucleic acid sequencing technology is not entirely error-free, SEQ ID NO:1 (as well as other sequences presented herein), at best, represents an apparent nucleic acid sequence of the polynucleotide encoding an S. aureus MRS polypeptide of the present invention.

In another embodiment, an S. aureus MRS gene can be an allelic variant that includes a similar but not identical sequence to SEQ ID NO:1. An allelic valiant of an S. aureus MRS gene including SEQ ID NO:1 is a locus (or loci) in the genome whose activity is concerned with the same biochemical or developmental processes, and/or a gene that that occurs at essentially the same locus as the gene including SEQ ID NO:1, but which, due to natural variations caused by, for example, mutation or recombination, has a similar but not identical sequence. Because genomes can undergo rearrangement, the physical arrangement of alleles is not always the same. Allelic variants typically encode polypeptides having similar activity to that of the polypeptide encoded by the gene to which they are being compared. Allelic variants can also comprise alterations in the 5′ or 3′ untranslated regions of the gene (e.g., in regulatory control regions). Allelic variants are well known to those skilled in the art and would be expected to be found within a given bacterial strain and/or among a population comprising two or more bacterial strains.

According to the present invention, an isolated, or biologically pure, polypeptide, is a polypeptide that has been removed from its natural milieu. As such, “isolated” and “biologically pure” do not necessarily reflect the extent to which the polypeptide has been purified. An isolated MRS polypeptide of the present invention can be obtained from its natural source, can be produced using recombinant DNA technology or can be produced by chemical synthesis. An MRS polypeptide of the present invention may be identified by its ability to perform the function of natural MRS polypeptide a functional assay. The wild type MRS protein catalyzes the covalent joining of the amino acid methionine to its specific tRNA molecule. This activity can be assayed and characterized by methods known to those of skill in the art. By “natural MRS polypeptide,” it is meant the full length MRS polypeptide of S. aureus. The phrase “capable of performing the function of a natural MRS polypeptide in a functional assay” means that the polypeptide has at least about 10% of the activity of the natural polypeptide in the functional assay. In other embodiments, the MRS polypeptide has at least about 20% of the activity of the natural polypeptide in the functional assay. In other embodiments, the MRS polypeptide has at least about 30% of the activity of the natural polypeptide in the functional assay. In other embodiments, the MRS polypeptide has at least about 40% of the activity of the natural polypeptide in the functional assay. In other embodiments, the MRS polypeptide has at least about 50% of the activity of the natural polypeptide in the functional assay. In other embodiments, the polypeptide has at least about 60% of the activity of the natural polypeptide in the functional assay. In other embodiments, the polypeptide has at least about 70% of the activity of the natural polypeptide in the functional assay. In other embodiments, the polypeptide has at least about 80% of the activity of the natural polypeptide in the functional assay. In other embodiments, the polypeptide has at least about 90% of the activity of the natural polypeptide in the functional assay. Examples of functional assays include antibody-binding assays, or aminoacylation assays, as detailed elsewhere in this specification.

A mutant MRS protein is a protein that has at least one change in an amino acid compared with a protein substantially corresponding to a wild type MRS protein. The change can be an amino acid substitution, deletion, or addition. There can be more than one change in the amino acid sequence, including 1 to 6 changes. In some embodiments, there is more than one change. For mutant MRS proteins useful in vaccines, it is desirable that the change in the amino acid sequence of the protein does not result in a change of the protein's ability to stimulate an antibody response that can neutralize wild type MRS protein. For mutant MRS proteins useful in vaccines, it is useful if the mutant proteins are recognized by polyclonal neutralizing antibodies to MRS protein.

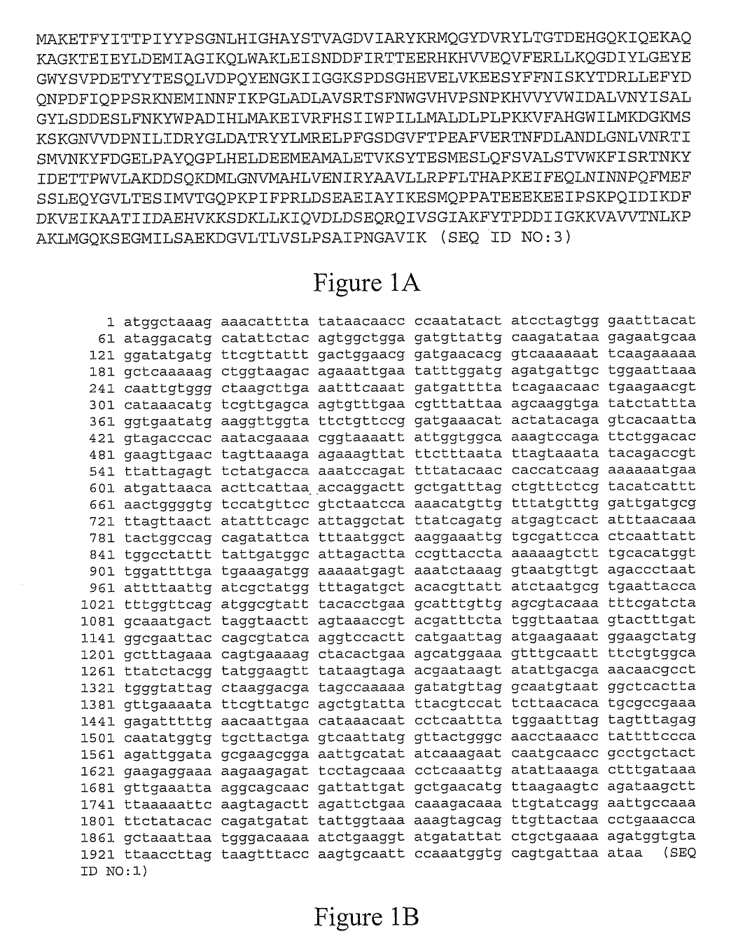

Changes in the amino acid sequence at a particular site can be randomly made or specific changes can be selected. Once a specific site is selected it is referred to by the amino acid found at that site in the mutant MRS protein, followed by its amino acid number designation and by the amino acid found at that site in the wild type MRS protein as shown in FIG. 1A. The amino acid number designations made in this application are by reference to the sequence in GenBank BAB41678 (ORFID:SA0448). Equivalent residues include those found in homologous molecules that can be identified as equivalent to amino acids in proteins substantially corresponding to a wild type MRS protein either by comparison of primary amino acid structure or by comparison to a modelled structure or by comparison to a known crystal structure of a homologous molecule. It is intended that the invention cover changes to equivalent amino acids at the same or similar locations regardless of their amino acid number designation.

Substitutions at a specific site can also include but are not limited to substitutions with non-naturally occurring amino acids such as 3-hydroxyproline, 4-hydroxyproline, homocysteine, 2-aminoadipic acid, 2-aminopimilic acid, ornithine, homoarginine, N-methyllysine, dimethyl lysine, trimethyl lysine, 2,3-diaminopropionic acid, 2,4-diaminobutryic acid, hydroxylysine, substituted phenylalanine, norleucine, norvaline, .gamma.-valine and halogenated tyrosines. Substitutions at a specific site can also include the use of analogs which use non-peptide chemistry including but not limited to ester, ether and phosphoryl and boron linkages.

The mutant polypeptides can be generated using a variety of methods. Those methods include site-specific mutagenesis, mutagenesis methods using chemicals such as EMS, or sodium bisulfite or UV irradiation, by spontaneous mutation, by in vitro mutagenesis and chemical synthesis. Methods of mutagenesis can be found in Sambrook et al., A GUIDE TO MOLECULAR CLONING, Cold Spring Harvard, New York (1989).

In one embodiment, the amino acid change is an amino acid change which is associated with decreased susceptibility to Compound 1.

In some embodiments, a mutant MRS protein in S. aureus is protein in which the amino acid change is an amino acid change comprising one or more of the following amino acid changes: L19F, T50A, E52D, G54S, G54A, I57N, I57T, Q58L, A61V, A61T, A64P, A64S, A77V, E98G, I94N, R100S, V108M, V108L, I238F, L213W, V215A, V215I, G223C, P230T, I238F, V242F, V242I, A247E, L257P, M269I, and V296F.

The spontaneous resistance studies and the serial passages described herein produced a large number of S. aureus isolates with decreased susceptibility to Compound 1.

Mutants can be generated to affect a functional change by changing amino acids in a particular domain of a molecule, such as the as binding domain of MRS. While not meant to limit the invention, it is believed that these domains form specific 3-D conformations that are important in the biological functions of the wild type MRS activity. Attempts to obtain crystals of the S. aureus MRS have failed, however, crystallographic studies on the E. coli MRS have been more successful, both in native form and as a complex with methionine. The hydrophobic binding pocket consists of 10 amino acids surrounding the L-methionine, and the amine group of L-methionine is hydrogen bonded to the carboxyl group of Asp52 and the carbonyl oxygen atom of Leu13. In a study published by Kim and Lee in 2003, two analogs of Compound 1 were docked into the binding pocket of FE coli MRS using the comparative molecular field analysis method, and were found to form hydrogen bonds to Asp296 and to a water molecule. S. aureus contains a type 1-MRS, while E. coli has a type 2-MRS. The overall identity is only 26%, however, the two proteins are similar in length (657 amino acid residues in S. aureus MRS versus 677 residues in E. coli MRS) and share key residues implicated in substrate binding. Interestingly, the MRS mutations G54S and I57N that were identified as the most frequent changes leading to reduced susceptibility to Compound 1 are conserved between E. coli and S. aureus and are very near the Asp52 residue that forms a hydrogen bond to methionine in E. coli MRS. Moreover, many MRS mutations occur in the amino acid region 213-296, which is a domain located very near the binding pocket and centered around Val 252 that has been described to be in a conformational constraint in the E. coli enzyme upon inhibitor binding. For the energy minimization model for one of the representatives of this class (E. coli MRS), the compound 1 binding site overlaps the known binding site of methionine. This suggests that corresponding mutations in other bacterial MRS proteins are useful in the methods and assays described here, such as preparation of vaccines and use as a target in binding and activity assays. The model of the S. aureus MRS active-site was created using the known crystal structure of E. coli MRS apo form (PDB ID=1QQT) as a template. Strikingly, all amino acid substitutions among our collection of S. aureus MRS mutants with decreased susceptibility to Compound 1 are located around the active site. The amino acid changes I57N G54S (S. aureus mutant 6-01, MIC=32 μg/ml) and A61T A64S (S. aureus mutant SP-25F, MIC=16 μg/ml) clearly extend into the active site. Furthermore, the substitution of the small non-polar side-chains found in wild-type MRS with the bulkier and polar side-chains in these mutants most likely affects the hydrophobic pocket, leading to lower affinity for Compound 1 and thus decreased susceptibilities of the corresponding strains to this MRS inhibitor.

Mutants for vaccine compositions are mutant MRS polypeptides that immunoreact with polyclonal neutralizing antibodies to wild type MRS polypeptide are useful.

A large number of MRS mutations that reduced susceptibility to Compound 1 have been characterized and may prove useful to gain further insight in the drug-target interactions.

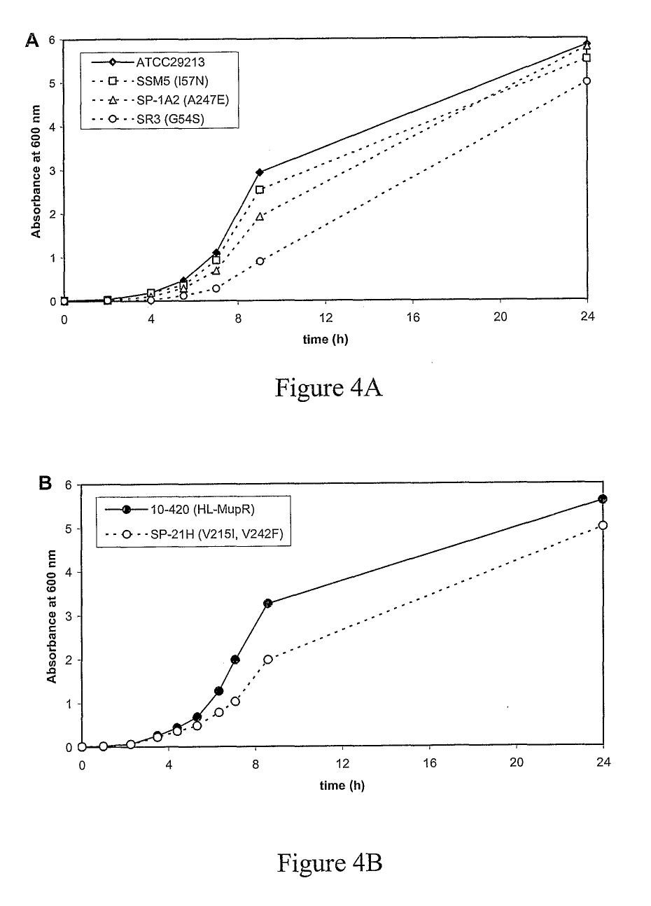

Only one of the key MRS mutations, G54S, was associated with a significant fitness burden. As used herein, a fitness burden refers to an extended lag phase and/or a reduced growth rate compared to the parental strain or other control. The effect of MRS mutations on fitness was assessed by monitoring growth in Mueller-Hinton broth (FIG. 4). S. aureus isolates harboring the single mutations I57N (MIC=4 μg/ml) or A247E (MIC=8 μg/ml) had a rather minor effect on growth. However, the single mutation leading to the highest MIC of 32 μg/ml, G54S, caused the most dramatic fitness burden, both an extended lag phase and a reduced growth rate compared to its parental strain (FIG. 4A). Similarly, the combination of the two key mutations V215I and V242F in strain SP-21H (MIC=32 μg/ml) also resulted in a reduced growth rate compared to its parent 10-420, which is a high-level mupirocin-resistant strain (FIG. 4B). In a report on mupirocin resistance, first-step mutants (V588F and V631F) in IRS were generally not associated with fitness costs, but second step mutants were unfit, however, fitness was restored by subculture in the absence of mupirocin as a result of compensatory mutations. Thus in one embodiment a mutant MRS protein is one which is associated with fitness burden.

Mutant MRS polypeptides are useful to form vaccine compositions. Mutants for vaccine compositions have at least one amino acid change, are nontoxic systemically, and immunoreact with polyclonal neutralizing antibodies to wild type MRS. Mutant proteins are combined with a physiologically acceptable carrier. Physiologically acceptable diluents include physiological saline solutions, and buffered saline solutions at neutral pH such as phosphate buffered saline. Other types of physiological carriers include liposomes or polymers and the like. Optionally, the mutant protein, can be combined with an adjuvant such as Freund's incomplete adjuvant, Freund's Complete adjuvant, alum, monophosphoryl lipid A, alum phosphate or hydroxide, QS-21 and the like. Optionally, the mutant proteins or fragments thereof can be combined with immunomodulators such as interleukins, interferons and the like. Vaccine formulations are known to those of skill in the art.

The mutant MRS protein or fragment thereof is added to a vaccine formulation in an amount effective to stimulate a protective immune response in an animal to at least one biological activity of wild type MRS protein. Generation of a protective immune response can be measured by the development of antibodies, preferably antibodies that neutralize the wild type MRS protein. Neutralization of wild type MRS protein can be measured including by inhibition of lethality due to wild type MRS in animals. In addition, a protective immune response can be detected by measuring a decrease in at least one biological activity of wild type MRS proteins such as amelioration or elimination of the symptoms of enhancement of endoprotein shock or STSS. The amounts of the mutant protein that can form a protective immune response are about 0.1 μg to 100 mg per kg of body weight more preferably about 1 μg to about 100 μg/kg body weight. About 25 μg/kg of body weight of wild type MRS protein is effective to induce protective immunity in rabbits. The vaccine compositions are administered to animals such as rabbits, rodents, horses, and humans.

The invention also includes fragments of mutant MRS proteins. For vaccine compositions, fragments are preferably large enough to stimulate a protective immune response. A minimum size for a B cell epitope is about 4-7 amino acids and for a T cell epitope about 8-12 amino acids. The total size of wild type MRS is about 657 amino acids. Fragments are peptides that are about 4 to 100 amino acids. In some embodiments, the fragment is 10-50 amino acids.

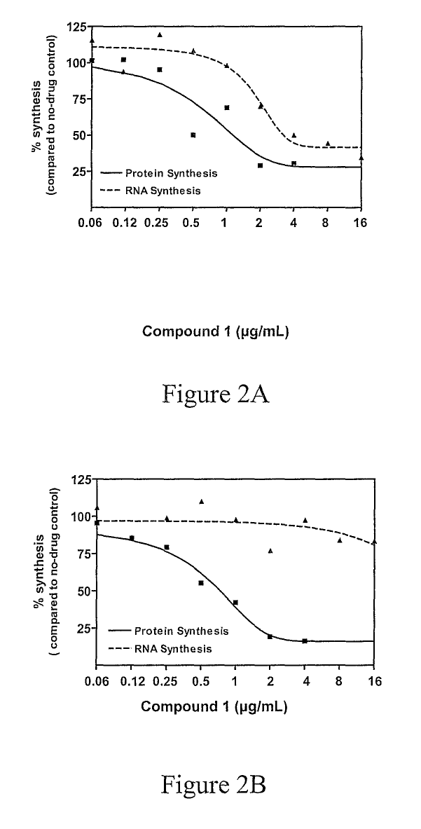

Fragments can be a single peptide or include peptides from different locations joined together. Fragments may include one or more of the domains as identified in FIG. 2 and as described previously. The fragments from mutant MRS proteins may also have at least one change in amino acid sequence and more preferably 1-6 changes in amino aced sequence when compared to a protein substantially corresponding to a wild type MRS protein.

Preferably, fragments are substantially nonlethal systemically. It is also preferred that the fragment is nontoxic in humans when given a dose comparable to that of a wild type MRS protein.

For vaccine compositions, the fragment stimulates a neutralizing antibody response to a protein having wild type MRS protein activity. A fragment can be screened and selected for immunoreactivity with polyclonal neutralizing antibodies to a wild type MRS protein. The fragments can also be used to immunize animals and the antibodies formed tested for neutralization of wild type MRS protein.

The fragments screened and selected for vaccine compositions can be combined into vaccine formulations and utilized as described previously. Optionally, fragments can be attached to carrier molecules such as bovine serum albumin, human serum albumin, keyhole limpet hemocyanin, tetanus toxoid and the like.

Fragments of mutant MRS protein can be prepared using PCR, restriction enzyme digestion and/or ligation, in vitro mutagenesis and chemical synthesis. For smaller fragments chemical synthesis may be desirable.

The mutant MRS proteins and/or fragments thereof are useful in methods for protecting animals against the effects of wild type MRS proteins, and ameliorating or treating animals with bacterial infections.

A method for protecting animals against at least one biological activity of wild type MRS protein involves the step of administering a vaccine composition to an animal to establish a protective immune response against at least one biological activity of MRS protein. It is preferred that the protective immune response is neutralizing and protects against lethality or symptoms of bacterial infection. The vaccine composition can be administered to an animal in a variety of ways including subcutaneously, intramuscularly, intravenously, intradermally, orally, intranasally, ocularly, intraperitoneally and the like. The vaccine composition can be administered in a single or multiple doses until protective immunity against at least one of the biological activities of wild type MRS is established. Protective immunity can be detected by measuring the presence of neutralizing antibodies to the wild type MRS using standard methods. An effective amount is administered to establish protective immunity without causing substantial toxicity.

A mutant MRS protein or fragment thereof is also useful to generate neutralizing antibodies that immunoreact with the mutant MRS protein and the wild type MRS protein. These antibodies could be used as a passive immune serum to treat or ameliorate the symptoms in those patients that have the symptoms of infection. A vaccine composition as described above could be administered to an animal such as a horse or a human until a neutralizing antibody response to wild type MRS is generated. These neutralizing antibodies can then be harvested, purified, and utilized to treat patients exhibiting symptoms of infection.

The neutralizing antibodies are administered to patients exhibiting symptoms of infection such as fever in an amount effective to neutralize the effect of MRS protein. The neutralizing antibodies can be administered intravenously, intramuscularly, intradermally, subcutaneously, and the like. In some embodiments, the neutralizing antibody be administered in conjunction with antibiotic therapy. The preferred amount of neutralizing antibodies typically administered is about 1 mg to 1000 mg/kg, and in some embodiments about 50-200 mg/kg of body weight.

The present invention is also directed to the identification of ligands, inhibitors or promoters of MRS function. Using the isolated mutant MRS proteins of the present invention, the present invention further provides methods of obtaining and identifying agents which are ligands, inhibitors, or promoters of bacterial MRS. In general, such methods comprise steps of: (a) contacting an agent with an isolated mutant MRS protein or fragment thereof, and (b) determining whether the agent binds to or modulates the function of said protein or said fragment. The agents screened in the above assay can be, but are not limited to, peptides, carbohydrates, vitamins, nucleic acids, botanically-derived compounds, organic compounds, pharmaceutical agents, and derivatives of the foregoing. As used herein, by “modulate the function,” it is meant altering when compared to not adding an agent. Modulation may occur on any level that affects function. A polynucleotide or polypeptide function may be direct or indirect, and measured directly or indirectly. Modulation may be an increase (stimulation) or a decrease (inhibition) in the function of the target.

The agents can be selected and screened at random or rationally selected or designed using protein modeling techniques. For random screening, agents such as peptides, carbohydrates, pharmaceutical agents and the like are selected at random and are assayed for their ability to bind or inhibit the MRS of the present invention. Alternatively, agents may be rationally selected or designed. As used herein, an agent is said to be “rationally selected or designed” when the agent is chosen based on the configuration of the particular protein.

As used herein, a ligand is a substance which binds to an MRS protein. In one embodiment, a ligand can bind selectively to two or more bacterial MRS proteins, including S. aureus MRS. In one embodiment, ligand binding of a mutant MRS protein occurs with high affinity. The term ligand refers to substances including, but not limited to, a natural ligand, whether isolated and/or purified, synthetic, and/or recombinant, a homolog of a natural ligand (e.g., from another mammal), antibodies, portions of such molecules, and other substances which bind the protein. A natural ligand of a selected bacterial MRS can bind to the protein under physiological conditions. The term ligand encompasses substances which are inhibitors or promoters of MRS, as well as substances which bind but lack inhibitor or promoter activity.

As used herein, an inhibitor is a substance which inhibits at least one function characteristic of an MRS protein, such as a binding activity (e.g., ligand, inhibitor and/or promoter binding), or a synthetase activity (e.g., covalent joining of an amino acid to its specific tRNA molecule). The term inhibitor refers to substances including antagonists which bind protein (e.g., an antibody, a mutant of a natural ligand, other competitive inhibitors of ligand binding), and substances which inhibit MRS function without binding thereto (e.g., an anti-idiotypic antibody).

As used herein, a promoter is a substance which promotes (induces or enhances) at least one function characteristic of a bacterial MRS protein, such as a binding activity or synthetase activity. The term promoter refers to substances including agonists which bind the protein (e.g., an antibody, a homolog of a natural ligand from another species), and substances which promote protein function without binding thereto (e.g., by activating an associated protein).

The assays described herein, which rely upon the nucleic acids and proteins of the present invention, can be used, alone or in combination with each other or other suitable methods, to identify ligands, inhibitors or promoters of a bacterial MRS protein or polypeptide. Cells which contain and express a mutant MRS protein of the present invention are useful in identifying ligands, inhibitors and promoters of MRS proteins.

Upon isolation of a mutant MRS gene from bacteria, the gene can be incorporated into an expression system to produce an MRS protein or polypeptide as described above. An isolated and/or recombinant MRS protein or polypeptide, such as a protein expressed in cells stably or transiently transfected with a construct comprising a nucleic acid of the present invention, or in a cell fraction containing protein, can be used in tests for MRS function. The protein can be further purified if desired. Testing of protein function can be carried out in vitro or in vivo.

An isolated, recombinant bacterial mutant MRS protein, can be used in the present method, in which the effect of a compound is assessed by monitoring MRS function as described herein or using other suitable techniques. See e.g., Example 2 and 3.

According to the method of the present invention, compounds can be individually screened or one or more compounds can be tested simultaneously according to the methods herein. Where a mixture of compounds is tested, the compounds selected by the processes described can be separated (as appropriate) and identified by suitable methods (e.g., PCR, sequencing, chromatography). The presence of one or more compounds (e.g., a ligand, inhibitor, promoter) in a test sample can also be determined according to these methods. Large libraries, including combinatorial libraries of compounds (e.g., organic compounds, recombinant or synthetic peptides, “peptoids”, nucleic acids) produced by combinatorial chemical synthesis or other methods can be tested.

The in vitro method of the present invention can be used in high-throughput screening. These assays can be adapted for processing large numbers of samples (e.g., a 96 well format). For such screening, a host cell expressing the mutant MRS protein, or isolated mutant MRS protein may be used.

The isolated and/or recombinant MRS proteins, portions thereof, or suitable fusion proteins of the present invention, can be used in a method to select and identify compounds which bind to a bacterial mutant MRS protein, such as S. aureus MRS, and which are ligands, or potential inhibitors or promoters of MRS activity. Compounds selected by the method, including ligands, inhibitors or promoters, can be further assessed for an inhibitory or stimulatory effect on MRS function and/or for therapeutic utility.

In one embodiment, compounds which bind to an active, isolated and/or recombinant mutant bacterial MRS protein or polypeptide are identified by the method. In this embodiment, the protein or polypeptide used has at least one function characteristic of a bacterial MRS protein. For example, an isolated and/or recombinant mutant bacterial MRS protein or polypeptide can be maintained under conditions suitable for binding, the protein is contacted with a compound to be tested, and binding is detected or measured. In one embodiment, the protein can be expressed in cells stably or transiently transfected with a construct comprising a nucleic acid sequence which encodes a mutant MRS protein of the present invention. The cells are maintained under conditions appropriate for expression of MRS. The cells are contacted with a compound under conditions suitable for binding (e.g., in a suitable binding buffer), and binding is detected by standard techniques. To measure binding, the extent of binding can be determined relative to a suitable control (e.g., compared with background determined in the absence of compound, compared with binding of a second compound (i.e., a standard), compared with binding of compound to normal cells). Optionally, a cellular fraction containing MRS can be used in lieu of whole cells.

In one embodiment, the compound is labeled with a suitable label (e.g., fluorescent label, isotope label), and binding is determined by detection of the label. Specificity of binding can be assessed by competition or displacement, for example, using unlabeled compound or a second ligand as competitor.

Ligands, including natural ligands from the same bacterial species or from another species, can be identified in this manner. The binding activity of a promoter or inhibitor which binds the protein can also be assessed using such a ligand binding assay.

Binding inhibition assays can also be used to identify ligands, and inhibitors and promoters which bind mutant MRS and inhibit binding of another compound such as a ligand. For example, a binding assay can be conducted in which a reduction in the binding of a first compound, such as an amino acid (in the absence of a second compound), as compared binding of the first compound in the presence of the second compound, is detected or measured. The protein can be contacted with the first and second compounds simultaneously, or one after the other, in either order. A reduction in the extent of binding of the first compound in the presence of the second compound, is indicative of inhibition of binding by the second compound. For example, binding of the first compound could be decreased or abolished.

In one embodiment, direct inhibition of the binding of a first compound (e.g., an amino acid) to a mutant MRS by a second test compound is monitored. For example, the ability of a compound to inhibit the binding of methionine to mutant bacterial MRS can be monitored. Such an assay can be conducted using either whole cells, a fraction from said cells, or isolated mutant MRS for instance.

Other methods of identifying the presence of a compound(s) which bind a mutant MRS compound are described herein.

It will be understood that the inhibitory effect of antibodies of the present invention can be assessed in a binding inhibition assay. Competition between antibodies for MRS binding can also be assessed in the method in which the first compound in the assay is another antibody, under conditions suitable for antibody binding.

Ligands, as well as protein-binding inhibitors (e.g., antagonists) and promoters (e.g., agonists), which are identified in this manner, can be further assessed to determine whether, subsequent to binding, they act to inhibit or activate other functions of bacterial MRS and/or to assess their therapeutic utility.

For completeness, various aspects of the invention are set out in the following numbered clauses:

1. An isolated mutant S. aureus MRS polypeptide comprising an isolated polypeptide having at least one amino acid changed relative to SEQ ID NO: 3 or a fragment of SEQ ID NO:3, and wherein the mutant S. aureus MRS polypeptide is associated with decreased susceptibility to an antagonist of the polypeptide.

2. The method of Clause 1, wherein the antagonist is N-(4-bromo-5-(1-fluorovinyl)-3-methylthiophen-2-ylmethyl)-N′-(1H-quinolin-4-one)propane-1,3-diamine.

3. The isolated S. aureus mutant MRS polypeptide of clause 1, wherein said isolated polypeptide comprises a mutation selected from the group consisting of L19F, T50A, E52D, G54S, G54A, I57N, I57T, Q58L, A61V, A61T, A64P, A64S, A77V, E98G, I94N, R100S, V108M, V108L, I238F, L213W, V215A, V215I, G223C, P230T, I238F, V242F, V242I, A247E, L257P, M269I, and V296F, and any combination of the foregoing.

4. The isolated S. aureus mutant MRS polypeptide of clause 1, wherein said isolated polypeptide exhibits at least one change in a biological function compared with a protein substantially corresponding to a wild type MRS protein.

5. The isolated S. aureus mutant MRS polypeptide of clause 1, wherein said change is selected from the group consisting of reduction of synthetase activity and increase in fitness burden.

6. A method, comprising

(a) contacting a compound to be tested with an isolated mutant MRS polypeptide; and

(b) determining whether the compound is a ligand of said polypeptide, wherein formation of a complex between the compound and the polypeptide is indicative that the compound is a ligand.

7. The method of clause 6, wherein the compound to be tested is selected from the group consisting of peptides, carbohydrates, vitamins, nucleic acids, botanically-derived compounds, organic compounds, pharmaceutical agents.

8. The method of clause 6, wherein the mutant bacterial MRS polypeptide is a mutant S. aureus MRS polypeptide.

9. The method of clause 8, wherein the mutant S. aureus MRS polypeptide is associated with decreased susceptibility to N-(4-bromo-5-(1-fluorovinyl)-3-methylthiophen-2-ylmethyl)-N′-(1H-quinolin-4-one)propane-1,3-diamine.

10. The method of clause 9, wherein the mutant S. aureus MRS polypeptide comprises a mutation selected from the group consisting of L19F, T50A, E52D, G54S, G54A, I57N, I57T, Q58L, A61V, A61T, A64P, A64S, A77V, E98G, I94N, R100S, V108M, V108L, I238F, L213W, V215A, V215I, G223C, P230T, I238F, V242F, V242I, A247E, L257P, M269I, and V296F, and any combination of the foregoing.

11. The method of clause 6, wherein the mutant bacterial MRS polypeptide is selected from the group consisting of a full length polypeptide and a fragment of a full length polypeptide.

12. The method of clause 6, wherein the formation of a complex is monitored by detecting or measuring a synthetase activity or cellular response by said mutant MRS protein in response to binding of a ligand thereto.

13. A method comprising

a) contacting compound to be tested with an isolated mutant MRS polypeptide with methionine under conditions suitable for binding of methionine to said polypeptide; and

b) detecting or measuring the formation of a complex between said polypeptide and methionine; and

c) determining whether the compound is a ligand said polypeptide, wherein inhibition of complex formation by the compound is indicative that the compound is a ligand of the polypeptide.

14. The method of clause 13, wherein said methionine is labeled with a detectable label.

15. The method of clause 13, wherein the inhibition of complex formation is monitored by detecting or measuring a decrease in synthetase activity or cellular response by said mutant MRS protein in response to binding of a ligand thereto.

16. The method of clause 13, wherein the compound to be tested is selected from the group consisting of peptides, carbohydrates, vitamins, nucleic acids, botanically-derived compounds, organic compounds, pharmaceutical agents.

17. The method of clause 13, wherein the mutant bacterial MRS polypeptide is a mutant S. aureus MRS polypeptide.

18. The method of clause 17, wherein the mutant S. aureus MRS polypeptide is associated with decreased susceptibility to N-(4-bromo-5-(1-fluorovinyl)-3-methylthiophen-2-ylmethyl)-N′-(1H-quinolin-4-one)propane-1,3-diamine.

19. The method of clause 18, wherein the mutant S. aureus MRS polypeptide comprises a mutation selected from the group consisting of L19F, T50A, E52D, G54S, G54A, I57N, I57T, Q58L, A61V, A61T, A64P, A64S, A77V, E98G, I94N, R100S, V108M, V108L, I238F, L213W, V215A, V215I, G223C, P230T, I238F, V242F, V242I, A247E, L257P, M269I, and V296F, and any combination of the foregoing.

20. The method of clause 13, wherein the mutant bacterial MRS polypeptide is selected from the group consisting of a full length polypeptide and a fragment of a full length polypeptide.

### EXAMPLES

The following examples are provided for illustrative purposes only and are not intended to limit the scope of the invention.

### Example 1

### Strains, Plasmids, Media, and Chemicals

S. aureus and S. pyogenes strains for Compound 1 resistance evaluation were obtained from ATCC, NARSA, and Focus Technologies. S. aureus strain RN4220 containing the plasmid pYH4-MRS and the S. pneumoniae strains R6 and R6 spo rel were provided by GlaxoSmithKline. Mueller-Hinton broth (MHB), Mueller-Hinton agar (MHA), and blood agar plates were from Remel (Lenexa, Kans.). Streptococci were grown in MHB containing 3% lysed horse blood (MHB/LHB). Stock cultures were prepared in Brucella broth with 20% glycerol (Hardy Diagnostics, Santa Maria, Calif.). Purified preparations of S. aureus MetS, H influenzae MetG, E. coli MetG, and S. pneumoniae MetS2 enzymes, as well as a preparation of rat liver lysate, were all provided by GlaxoSmithKline. E. coli MRE 600 tRNA was from Roche Applied Science (Indianapolis, Ind.).

Compound 1 was synthesized at Replidyne, Inc. Mupirocin was provided by Pliva (Zagreb, Croatia). Novobiocin (USP), gentamicin (Sigma) and vancomycin (USP) were used as control agents. Anhydrotetracycline was purchased from BD Biosciences (Palo Alto, Calif.). The radiolabeled compounds [5-³H]uridine, L-[4,5-³H]leucine, and L-[methyl-³H]methionine were from Amersham Biosciences Corp. (Piscataway, N.J.).

### Example 2

### MRS Enzymatic Assay

Compound 1 is a potent, nanomolar inhibitor of S. aureus MetS (Table 1).

TABLE 1
Inhibition of MetS Activity by Compound 1

| MRS Enzyme Activity | IC50^{a} | |
|---|---|---|
| S. aureus MetS | <1.9 | nM |
| H influenzae MetG | 25 | nM |
| E. coli MetG | 307 | nM |
| S. pneumoniae MetS2 | >500 | nM |
| Rat liver lysate^{b} | >500 | nM |

^{a}The specific activity of individual enzyme preparations varied. The enzyme concentrations were 1.5 nM for S. aureus, 3.0 nM for H. influenzae, 2.0 nM for E. coli and 12 nM for S. pneumoniae MRS. Enzyme concentrations were adjusted to achieve charging of 15-25 pmol of tRNA^{Met} in 15 min at 23° C.
^{b}Rat MRS concentration was not determined; the rat liver lysate catalyzed the charging of 5 pmol of tRNA^{Met} in 15 min at 23° C.

The IC50 for inhibition was as low as the concentration of enzyme present in the assay. Furthermore, the correlation between IC50 and enzyme concentration remained consistent as enzyme concentration was varied. This behavior was noted previously for similar S. aureus MetS inhibitors and indicates that measurement of a true inhibitory constant is limited by the inability to carry out the assay at lower enzyme concentrations. At present it can be concluded at a minimum that the IC50 for Compound 1 inhibition of S. aureus MetS is <1.9 nM. Compound 1 also inhibited two related MRS enzymes, H influenzae MetG and E. coli MetG (Table 1). These studies were carried out with enzyme concentrations that yielded activity within the linear range of the assay. Due to varying specific activities for the purified proteins from each different organism, a distinct enzyme concentration was used for each preparation. However, in contrast to the result for S. aureus MetS, the IC50 for Compound 1 inhibition was significantly higher than the enzyme concentrations used in the assay for both the H influenzae MetG (13-fold) and E. Coli MetG (160-fold) enzymes.

A recent study identified a gene that encodes a second methionyl-tRNA synthetase, metS2, in some strains of S. pneumoniae. The MetS2 gene was found to be present in 46% of 315 S. pneumoniae clinical isolates tested. Interestingly, the S. pneumoniae MetS2 protein was found to be resistant to inhibition by potent inhibitors of S. aureus MetS. Inhibition by Compound 1 was also tested against S. pneumoniae MetS2. Consistent with previous results, S. pneumoniae MetS2 was resistant to inhibition by Compound 1 (Table 1). The inhibition of a mammalian MRS activity by Compound 1 was also determined using MRS activity present in a rat liver lysate. No inhibition was observed for concentrations of Compound 1 of up to 500 nM (Table 1).

Assays for inhibition of tRNA^{Met} aminoacylation were carried out much as described by Macarrón et al., 2000 Anal. Biochem. 284:183-190. Reactions (50 μl) contained 65 mM Tris-HCl, pH 8.0, 80 mM KCl, 10 mM magnesium acetate, 2.5 mM dithiothreitol, 5 mM ATP, 1 mg/ml E. coli tRNA, 0.25 mg/ml bovine serum albumin, 7 μM methionine (specific activity 1.4 μCi/nmol) and were performed in round-bottom 96-well polypropylene plates (Costar). MRS enzymes were diluted in 50 mM Tris-HCl, pH 8.0, 2 mM dithiothreitol, 0.3 mg/ml bovine serum albumin and added (26 μl) to wells containing Compound 1 (4 μl) in DMSO or to control wells containing 4 μl DMSO or 0.5 M EDTA. A cofactor mix containing the remaining reaction components (20 μl) was added and plates were incubated for 15 min at room temperature (23° C.). Reactions were terminated and the tRNA was precipitated by the addition of 150 μl 5% trichloroacetic acid (TCA). Reaction mixtures were transferred to 96-well plates (Durapore, #MVHBN4550, Millipore, Bedford, Mass.) and filtered using a Manifold Filtration System from Innovative Microplate (Chicopee, Mass.). Samples were washed with 300 μl 10% TCA followed by 300 μl 95% ethanol and dried overnight. Reaction products were counted by liquid scintillation using MicroScint (50 μl) and a TopCount-NXT (Packard BioScience).

### Example 3

### Microbiological Assays

Broth microdilution MIC testing was performed in 96-well microtiter plates according to CLSI document M7-A6 (Clinical and Laboratory Standards Institute. 2005. Performance standards for antimicrobial susceptibility testing; fifteenth informational supplement. Villanova, Pa.). MIC testing of the metS overexpressor strain S. aureus RN4220 (pYH4-MRS) occurred in the presence or absence of the inducer anhydrotetracycline (10 ng/ml) to regulate expression of metS from the plasmid-borne TetR-dependent promoter. The macromolecular synthesis assays in S. pneumoniae R6 and an isogenic spo rel mutant were performed as follows. The cells were grown statically for 6 h at 35° C. in 10-ml MHB/LHB and the cell density was adjusted to match the 0.5 McFarland standard (approximately 10⁸ CFU/ml). A 96-well microtiter plate containing 50 μl of serially diluted Compound 1 at concentrations ranging from 16 μg/ml to 0.008 μg/ml was prepared and inoculated with 50 μl of cells. After incubation for 10 min at 35° C., the radiolabeled precursors [5-³H]uridine and L-[4,5-³H]leucine were added. Incorporation was stopped after 10 min by the addition of 100 μl of 20% ice-cold TCA. The plates were refrigerated for 1 h to allow for cell lysis and precipitation of macromolecules, followed by filtration through 96-well Durapore filter plates (MHVBN4550 from Millipore, Bedford, Mass.) using a vacuum manifold. The filter plates were washed twice with 200 μl of 10% ice-cold TCA, then once with 200 μl of cold ethanol, and air-dried. Microscint 0 (Perkin Elmer, Boston, Mass.) was added (50 μl), the plates were sealed and counted in a Packard TopCount NXT. The data were normalized to untreated controls and expressed as percentage of incorporation for each drug concentration.

The influence of MRS overexpression on the MIC of Compound 1 and control agents is shown in Table 2.

TABLE 2
MIC changes in S. aureus upon overexpression of the MRS target

| | S. aureus RN4220 MIC (μg/ml) | | | |
|---|---|---|---|---|
| Antimicrobial | pYH4 vector control | | pYH4-MRS | |
| agent | −aTc inducer | +aTc inducer | −aTc inducer | +aTc inducer |
| Compound 1 | 0.008 | 0.008 | 0.12 | 1 |
| Mupirocin | 0.06 | 0.06 | 0.06 | 0.06 |
| Novobiocin | 0.25 | 0.25 | 0.25 | 0.25 |
| Vancomycin | 1 | 1 | 0.5 | 0.5 |

Induction of MRS expression in S. aureus RN4220 (pYH4-MRS) resulted in an 8-fold shift in the MIC for Compound 1 from 0.12 μg/ml to 1 μg/ml. Interestingly, even the uninduced S. aureus RN4220 (pYH4-MRS) was significantly less susceptible to Compound 1 compared to the S. aureus RN4220 (pYH4) vector control strain (MIC=0.12 μg/ml versus MIC=0.008 μg/ml), indicating that plasmid pYH4-MRS may cause some expression of metS even in the absence of inducer. The MIC values for the control drugs mupirocin, novobiocin, and vancomycin remained unchanged upon MRS overexpression. These data provide good evidence that Compound 1 exerts its antibacterial activity through specific inhibition of MRS in S. aureus.

Second, macromolecular synthesis assays in S. pneumoniae in the presence of Compound 1 demonstrated a dose-dependent inhibition of protein and RNA synthesis, as expected with compounds that elicit stringent response (FIG. 2A). However, only protein synthesis was affected in a rel mutant deficient in stringent response (FIG. 2B). These data provide direct evidence that Compound 1 is a specific inhibitor of protein synthesis.

### Example 4

### Spontaneous Resistance Rates and MPC Determination

S. aureus was grown in MHB for 4-6 h at 35° C. The cells were harvested by centrifugation (4,000 rpm, 10 min) and resuspended in MHB at about 10¹⁰ CFU/ml. Agar plates containing Compound 1 or mupirocin at 2-fold increasing concentrations were seeded with 0.1 ml of this cell suspension (approximately 10⁹ CFU) and incubated at 35° C. The exact inoculum was determined by plating dilutions of the cell suspension on MHA and enumeration of colonies. Drug-containing MHA agar plates were scored for bacterial colonies after 48 h and the spontaneous resistance rates were calculated by dividing the number of colonies by the exact number of CFUs in the inoculum. These first-step mutants were purified on MHA containing Compound 1 or mupirocin at half the concentration of the original selection plate. Second-step mutants were isolated by repeating the procedure above with a first-step isolate as the parental strain. The stability of these isolates was assessed after five serial drug-free purification plates. To determine the MPC, the procedure was scaled up 10-fold by seeding five plates at each drug concentration with 2×10⁹ CFU to achieve an inoculum of at least 10¹⁰ CFU, and plates were scored for colonies after five days of incubation. The MPC was defined as the lowest drug concentration that prevented any emerging colonies from an inoculum of 10¹⁰ CFU after five days.

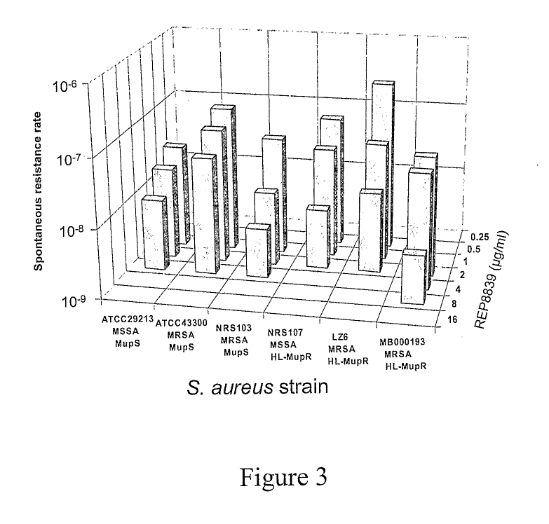

All tested strains, including MRSA and mupirocin-resistant isolates, were susceptible to Compound 1 with an initial MIC range of 0.03-0.5 μg/ml, but most strains gave rise to first-step resistant colonies on agar containing 4 μg/ml of Compound 1. The strain MB000193, which possessed the least initial susceptibility (MIC=0.5 μg/ml), was capable of forming colonies on agar with 8 μg/ml of Compound 1. The calculated resistance rates from a population of approximately 10⁹ cells were in the order of 10⁻⁷ to 10⁻⁸ after 48 h (FIG. 3). To further investigate the emergence of resistance, the Mutant Prevention Concentration (MPC) was determined, which is defined as the minimal drug concentration that prevents the emergence of spontaneous resistant subpopulations from 10¹⁰ cells within five days. The MPC was determined for S. aureus, including MSSA and MRSA strains, and was 32 μg/ml for Compound 1 and ≧32 μg/ml for mupirocin (Table 3).

TABLE 3
Mutation Prevention Concentration for Compound 1 and
mupirocin in S. aureus

| | MIC (μg/ml) | | MPC (μg/ml) | |
|---|---|---|---|---|
| S. aureus strain | Compound 1 | Mupirocin | Compound 1 | Mupirocin |
| ATCC 29213 | 0.03 | 0.12 | 32 | 32 |
| (MSSA) | | | | |
| ATCC 43300 | 0.06 | 0.12 | 32 | 32 |
| (MRSA) | | | | |
| LZ10 (MRSA) | 0.03 | 0.12 | 32 | >32 |
| 1079077 (MRSA) | 0.25 | 0.25 | 32 | >32 |

Also, the generation of second-step mutants was investigated by exposure of first-step mutants to even higher concentrations of Compound 1. Three first-step mutants that contained different point mutations in metS and had elevated MIC's of 4-8 μg/ml were in fact capable of producing second step mutants with new MIC=32 μg/ml (see characterization below). The second step spontaneous resistance rates were 2.4×10⁻⁸-2.2×10⁻⁹.

### Example 5

### Serial Passages

Changes in the susceptibilities of bacteria to Compound 1 were monitored upon continued exposure to near-MIC levels in serial passages in drug-containing broth. The first passage was a standard broth microdilution MIC test to record the initial MIC values. For the subsequent passages, the cells growing in the well with the highest inhibitor concentration, typically one dilution lower than the MIC, were resuspended in the well, diluted 1000-fold into broth, and used to inoculate a fresh 96-well MIC plate containing appropriate dilutions of the antibacterial agents. The MIC values of a total of 20 daily passages were recorded. The isolate after passage 20 was cultured on a purity plate and the final MIC was determined according to NCCLS guidelines.

Twenty serial passages caused the MIC for Compound 1 to shift from an initial range of 0.015-0.06 μg/ml to 0.06-16 μg/ml, particularly, there were five strains with MIC=16 μg/ml after 20 passages (Table 4).

TABLE 4
MIC changes in staphylococci and streptococci upon serial passages in broth
containing Compound 1 or mupirocin.

| | | Compound 1 | | Mupirocin | |
|---|---|---|---|---|---|
| | | MIC (μg/ml) | | MIC (μg/ml) | |
| | | | After 20 | | After 20 |
| Strain | Phenotype^{a} | Initial | passages | Initial | passages |
| S. aureus ATCC29213 | MSSA, MupS | 0.06 | 4 | 0.06 | 0.5 |
| S. aureus ATCC43300 | MRSA, MupS | 0.06 | 1 | 0.06 | 8 |
| S. aureus LZ10 | MRSA, MupS | 0.03 | 0.5 | 0.25 | 2 |
| S. aureus NRS103 | MRSA, MupS | 0.12 | 1 | 0.25 | 1 |
| S. aureus 1079077 | MRSA, MupS | 0.25 | 16 | 0.5 | 1 |
| S. aureus 31-1334 | MRSA, LL-MupR | 0.03 | 8 | 32 | 128 |
| S. aureus 1079101 | MSSA, HL-MupR | 0.06 | 16 | 4,096 | 4,096 |
| S. aureus NRS107 | MSSA, HL-MupR | 0.015 | 0.5 | 4,096 | 4,096 |
| S. aureus LZ1 | MRSA, HL-MupR | 0.06 | 0.06 | 4,096 | 4,096 |
| S. aureus LZ6 | MRSA, HL-MupR | 0.06 | 2 | 4,096 | 4,096 |
| S. aureus 10-420 | MRSA, HL-MupR | 0.06 | 8 | 4,096 | 4,096 |
| S. aureus 87-2797 | MRSA, HL-MupR | 0.03 | 16 | 2,048 | 4,096 |
| S. aureus 25-670 | MRSA, HL-MupR | 0.06 | 8 | 2,048 | 4,096 |
| S. epidermidis NRS8 | MRSE, LL-MupR | 0.06 | 0.12 | 32 | 128 |
| S. epidermidis 936528 | MRSE, HL-MupR | 0.03 | 0.5 | 2,048 | 4,096 |
| S. epidermidis 936606 | MRSE, MupS | 0.06 | 16 | 0.12 | 2 |
| S. hemolyticus NRS116 | MRSH, MupS | 0.12 | 16 | 0.12 | 1 |
| S. pyogenes ATCC19615 | EryS | 0.12 | 1 | 0.06 | 4 |
| S. pyogenes MB000143 | EryR | 0.12 | 1 | 0.06 | 0.5 |

^{a}MupS, mupirocin-susceptible; LL-MupR, low-level mupirocin-resistant; HL-MupR, high-level mupirocin-resistant

There was no correlation between the level of decreased susceptibility to Compound 1 and the oxacillin- or mupirocin-resistance phenotype of the individual staphylococcal strains. In S. pyogenes the effect of passaging was somewhat less dramatic than in staphylococci, with an MIC increase from 0.12 μg/ml to 1 μg/ml, and was not linked to the macrolide-resistance status. Mupirocin, which was used as a control and comparator drug, produced a similar shift of the MIC range from 0.06-0.5 μg/ml to 0.5-8 μg/ml in mupirocin-susceptible strains, and a shift from 32 μg/ml to 128 μg/ml in low-level mupirocin-resistant strains.

All of the laboratory-generated Compound I-resistant mutants were still susceptible to mupirocin, indicating that there is no cross-resistance between Compound 1 and mupirocin.

### Example 6

### Characterization of Mutants with Reduced Susceptibility to Compound 1

The MIC of resistant isolates was determined side by side with the original parental strain and stock cultures of the resistant isolates were prepared. Genomic DNA for molecular analysis was isolated from S. aureus using the DNeasy tissue kit (Qiagen Inc., Valencia, Calif.). A 1,067-bp fragment comprising the 5′-portion of the metS gene was amplified by PCR with high-fidelity PCR Supermix (Invitrogen, Carlsbad, Calif.) using primers metS1 (5′ACATTACGAGGAGGAACAG) (SEQ ID NO:4) and metS2 (5′-GGTGTAAATACGCCATCTG) (SEQ ID NO:5). The 3′-portion of metS was amplified with primers metS3 (5-GTCTTTGCACATGGTTGGA) (SEQ ID NO:6) and metS4 (5′-TGCTTCTCTAGCACGTGTA) (SEQ ID NO:7), yielding a 1,203-bp product. The PCR protocol for both fragments consisted of initial denaturation (5 min at 94° C.), 30 cycles of denaturation (1.5 min at 94° C.), annealing (1 min at 55° C.), and extension (1.5 min at 72° C.), followed by incubation for 10 min at 72° C. in a Techgene thermal cycler (Techne, Princeton, N.J.). PCR products were analyzed on a 0.8% agaraose/TBE gel, cloned into pCR® 4-TOPO (Invitrogen, Carlsbad, Calif.), and sequenced with T7 and T3 primers (Molecular Biology Core Facility, Barbara Davis Center for Childhood Diabetes, UCHSC, Denver, Colo.). Independent duplicate PCR fragments were processed to minimize PCR and DNA sequencing errors. The complete metS DNA sequences were assembled and aligned using Vector NTI (InforMax, Bethesda, Md.).

The spontaneous resistance studies and the serial passages had produced a large number of S. aureus isolates with decreased susceptibility to Compound 1. A total of 89 such strains were further characterized by DNA sequence analysis of the metS gene to reveal point mutations that lead to amino acid substitutions within MRS (Table 5).

TABLE 5
Molecular genetic characterization of laboratory-generated S. aureus mutants with
decreased susceptibility to Compound 1.

| Isolation method | Isolate # or ID | Parental strain | MIC (μg/ml) | MRS mutation(s) |
|---|---|---|---|---|
| Spontaneous | 25 isolates | ATCC 29213 | 4-8 | I57N |
| resistance | | | | |
| | SR5-1, SR5-2 | ATCC43300 (MRSA) | 8 | I57N |
| | 6 isolates | 87-2797 (MRSA, HL-MupR) | 4-16 | I57N |
| | SR23-1 | LZ10 (MRSA) | 8 | I57N |
| | SR59-1, SR59-2 | 1079077 (MRSA) | 8 | I57N |
| | SR74-2 | MB000193 | 8 | I57N |
| | SR3, SR18, SR21 | ATCC 29213 | 16-32 | G54S |
| | FSM7 | 87-2797 (MRSA, HL-MupR) | 16 | G54S |
| | SR74-1 | MB000193 | 16 | G54S |
| | SR84-1, SR84-2 | MB000057 | 16 | G54S |
| | SR23-2 | LZ10 (MRSA) | 32 | G54S |
| | SR19, SR23 | ATCC 29213 | 0.5-1 | none |
| | FSM8 | 87-2797 (MRSA, HL-MupR) | 1 | none |
| Serial passage | 8 isolates | ATCC 29213, others | 8-16 | I57N |
| | SP-21C | 31-1334 (MRSA, LL-MupR) | 8 | I57T |
| | SP-27H | 10-420 (MRSA, HL-MupR) | 4 | E52D |
| | SP-27D | 14-354 (MRSA, LL-MupR) | 2 | A61V |
| | SP-21A | ATCC 29213 | 4 | A77V |
| | SP-25G | LZ6 | 8 | V108M |
| | SP-2C4 | ATCC 43300 | 2 | L213W |
| | SP-28B | 25-670 (MRSA, HL-MupR) | 4 | V215A |
| | SP-4A2 | 87-2797 (MRSA, HL-MupR) | 8 | G223C |
| | SP-11A3 | 31-1334 (MRSA, LL-MupR) | 8 | I238F |
| | SP-1A2 | ATCC 29213 | 4 | A247E |
| | SP-9B5 | ATCC 29213 | 4 | I57N, V296F |
| | SP-2B5 | ATCC 43300 (MRSA) | 32 | I57N, R100S |
| | SP-4B5 | 87-2797 (MRSA, HL-MupR) | 32 | I57N, V242F |
| | SP-25C | 31-1334 (MRSA, LL-MupR) | 2 | T50A, V242I |
| | SP-26C | 1079101 | 32 | G54A, A64P |
| | SP-25F | LZ1 | 16 | A61T, A64S |
| | SP-22B | 25-670 (MRSA, HL-MupR) | 8 | A61T, V108L |
| | SP-21G | LZ6 | 2 | I94N, V215A |
| | SP-21H | 10-420 (MRSA, HL-MupR) | 8 | V215I, V242F |
| | SP-22C | 1079101 | 16 | L19F, Q58L, |
| | | | | A64P |
| | SP-25D | 14-354 (MRSA, LL-MupR) | 8 | A61T, E98G, |
| | | | | M269I |
| | SP-26B | 25-670 (MRSA, HL-MupR) | 4 | P230T, A247E, |
| | | | | L257P |
| | SP-21B | ATCC 43300 (MRSA) | 2 | none |
| | SP-3A5 | 31-1334 (MRSA, LL-MupR) | 2 | none |
| | SP-4D5 | 87-2797 (MRSA, HL-MupR) | 0.25 | none |
| Second step | 1-01, 1-02, 1-03 | ATCC 29213 | 32 | I57N, A247E |
| mutants | | | | |
| | 6-01 | ATCC 29213 | 32 | I57N, G54S |
| | 6-02, 6-03 | ATCC 29213 | 32 | I57N, I238F |
| | 14-01, 14-02 | LZ6 | 32 | G54S, V108M |

The vast majority of isolates obtained from spontaneous resistance experiments possessed an asparagine residue at position 57 instead of an isoleucine, and these I57N mutants had MIC values ranging from 4-16 μg/ml. The second type of spontaneous mutants harbored a G54S substitution in MRS, leading to MIC values ranging from 16-32 μg/ml. These G54S mutants formed tiny colonies on agar containing Compound 1, but gave rise to a large-colony-forming subpopulation on purity blood agar that was no longer resistant to Compound 1. In fact, such isolates were true revertants in which the glycine residue at position had been restored, as shown by sequencing the metS gene. The serial passages produced a larger variety of mutations in MRS, including double and triple mutations (Table 5). I57N mutants with MIC=8-16 μg/ml were still predominant, and also one I57T mutant (MIC=8 μg/ml) was isolated. Other mutants containing a single MRS mutation leading to MIC=2-8 μg/ml were E52D, A61V, A77V, V108M, L213W, V215, G223C, I238F, and A247E. Nine mutants harbored two changes in MRS, and three strains contained three changes, which often were combinations of individual mutations found in the single mutants. All second step mutants contained combinations of I57N or G54S with another key mutation already described above, and these were the most resistant strains, with MIC=32 μg/ml for Compound 1.

The characterization of MRS mutants with increased resistance to Compound 1 provides further evidence that Compound 1 is a specific inhibitor of MRS in S. aureus. Only a few spontaneous mutants or final isolates from serial passages did not have any alterations in MRS (e.g. SR19, SR23, FSM8, SP-21B, SP-3A, SP-4D), however, their susceptibilities to Compound 1 was only slightly decreased (MIC=0.25-2 μg/ml). This finding indicates that mechanisms other than MRS mutations, such as thicker cell walls, increasing the target copy number by higher expression levels, or efflux may only lead to minor decreases in susceptibility to Compound 1.

The effect of MRS mutations on fitness was assessed by monitoring growth in Mueller-Hinton broth (FIG. 4). S. aureus isolates harboring the single mutations I57N (MIC=4 μg/ml) or A247E (MIC=8 μg/ml) had a rather minor effect on growth. However, the single mutation leading to the highest MIC of 32 μg/ml, G54S, caused the most dramatic fitness burden, both an extended lag phase and a reduced growth rate compared to its parental strain (FIG. 4A). Similarly, the combination of the two key mutations V215I and V242F in strain SP-21H (MIC=32 μg/ml) also resulted in a reduced growth rate compared to its parent 10-420, which is a high-level mupirocin-resistant strain (FIG. 4B).

### Example 7

### Modeling of S. aureus MRS

Amino acid sequences for E. coli MRS apo form (PDB ID=1QQT) and co-crystallized form (PDB ID=1F4L) were derived from the PDB database. The WhatCheck protein verification tool (Hooft, et al., (1996) Nature 381:272) was used to assess the overall quality of the PDB files. Amino acid sequences for S. aureus wild-type and S. aureus mutants were obtained by translation of the corresponding metS DNA sequences (see above). Global pairwise amino acid sequence alignments for E. coli/S. aureus were generated with NCBI's Cn3D/MMDB sequence alignment tool (Hogue, 1997 Trends Biochem. Sci. 22:314-316, and Wang, et al. 2002. Nucleic Acids Res. 30:249-252.) The Needleman-Wunsch algorithm (Needleman, et al., 1970. J. Mol. Biol. 48:443-453) and the BLOSUM62 amino acid substitution scoring matrix were used for the alignments. Homology models were generated for S. aureus using E. coli MRS apo form (1 QQT) as the template structure. The homology models were created with MODELLER (Marti-Renom, et al. 2000. Annu. Rev. Biophys. Biomol. Struct. 29:291-325) by the satisfaction of spatial restraints (Sali, et al. 1993. J. Mol. Biol. 234:779-815) and the optimization of 3D structure using CHARMM force field energy terms. RMSD metrics were computed for the final 3D S. aureus structure relative to the E. coli template structure.

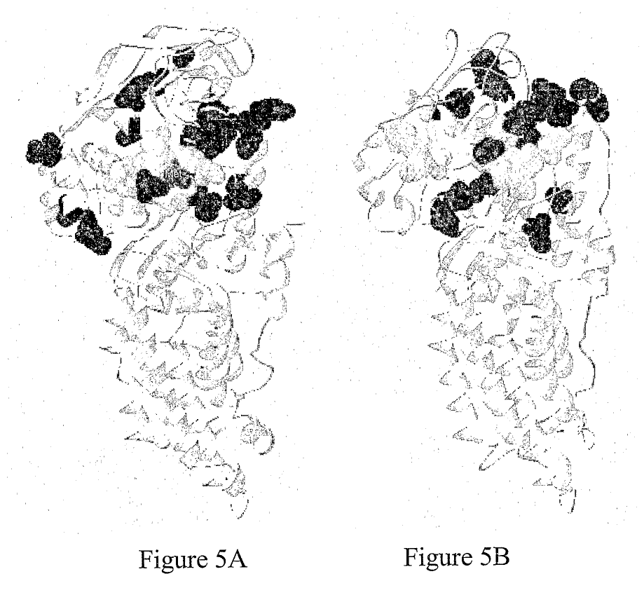

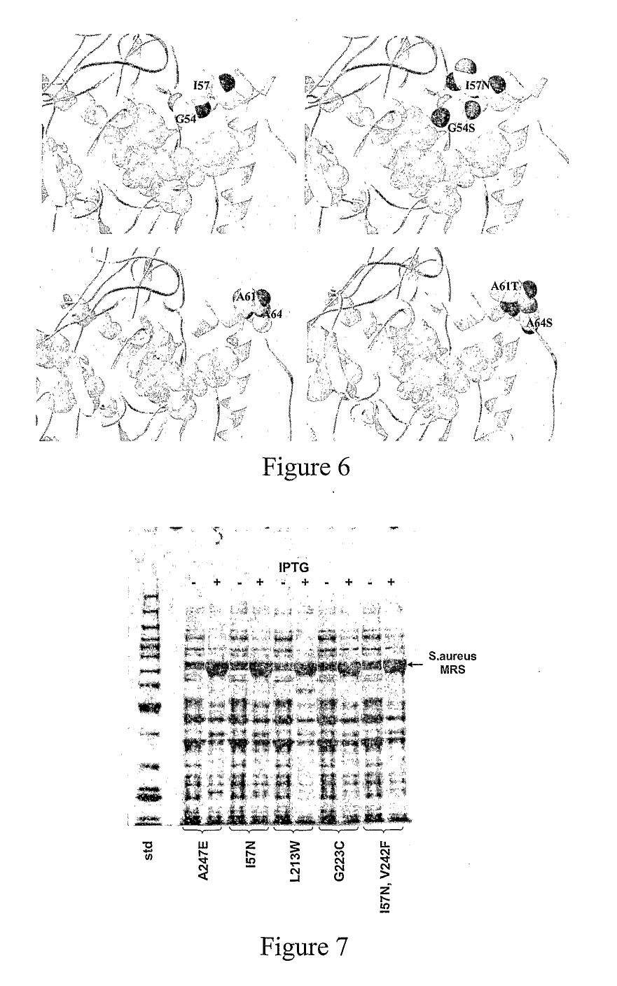

All mutations leading to a higher MIC for Compound 1 were located within the amino-terminal half of the MRS enzyme, which is in agreement with the fact that the carboxy-terminal portion contains the tRNA binding domain and thus seems unlikely to be affected by binding of Compound 1. The region from amino acid residues 50-64 was most frequently affected in MRS mutants and contains G54, I57, and A61, all of which are conserved within the six bacterial strains, and A64, which is conserved among strains harboring MRS type 1. Interestingly, several residues of altered S. aureus MRS had changed to the corresponding amino acids typically found at these positions in MRS type 2 enzymes (for which Compound 1 has a much lower affinity), such as 213W, 215I, and 296F. None of the mutations were located within the well conserved ATP-binding Rossman fold that contains the motifs H(M/I)GH and KMSKS that characterize all class I tRNA synthetases. A structural model of S. aureus MRS was generated based on the known E. coli MRS apo form (PDB ID=1QQT) and the amino acid sequence alignment that showed 38% similarity between the two proteins. The E. coli MRS and S. aureus MRS possess a considerably conserved 3D structure, as depicted in FIG. 5. All 23 amino acid substitutions found within mutant MRS proteins were found to be clustered around the active site where the ligand methionine is bound. The amino acid substitutions in mutant MRS proteins from S. aureus 6-01 (I57N G54S) and SP-25F (A61T, A64S) are located in close proximity to active site residues (FIG. 6). Their larger size and their hydrophilic nature may impair MRS function considerably.

### Example 8

### Biochemical Characterization of Mutants with Reduced Susceptibility to Compound 1

The IC50 and kinetic parameters of resistant isolates are determined side by side with the native S. aureus MRS. Genomic DNA for molecular analysis was isolated from S. aureus using the DNeasy tissue kit (Qiagen Inc., Valencia, Calif.) as described in Example 6. The entire 1974-bp metS gene was amplified by PCR with AccuPrime™ pfx DNA Polymerase (Invitrogen, Carlsbad, Calif.) using primers SAMRS-For-TOPO (5′CACCATGGCTAAAGAAACATTTTATATAACAAC) (SEQ ID NO:8) and SA-MRS-Rev2 (5′-GACTGTCGACTATTATTTAATCACTGCACCATTTG) (SEQ ID NO:9). The PCR protocol consisted of initial denaturation (2 min at 95° C.), 30 cycles of denaturation (0.5 min at 94° C.), annealing (1 min at 55° C.), and extension (2 min at 70° C.), followed by incubation for 5 min at 70° C. in a Techgene thermal cycler (Techne, Princeton, N.J.). PCR products were analyzed on a 0.8% agarose/TBE gel, cloned into pET101/D-TOPO and transformed into One Shot® TOP10 Competent Cells (Invitrogen, Carlsbad, Calif.) for plasmid amplification. Plasmids were isolated from 25 ml overnight cultures in Luria-Bertani (LB) medium containing 100 μg/ml ampicillin using Qiagen® (Plasmid Midi Kit (Qiagen Inc., Valencia, Calif.). The inserts were verified by NcoI/SpeI restriction digest screening and sequenced with metS2 (5′-GGTGTAAATACGCCATCTG) (SEQ ID NO:10), metS3 (5′-GTCTTTGCACATGGTTGGA) (SEQ ID NO:6), T7Rev2 (5′-TAGTTATTGCTCAGCGGTGG) (SEQ ID NO:11) and T7 promoter (5′-TAATACGACTCACTATAGGG) (SEQ ID NO:12) primers (Molecular Biology Core Facility, Barbara Davis Center for Childhood Diabetes, UCHSC, Denver, Colo.). Independent duplicate PCR products were processed to minimize PCR and DNA sequencing errors. The MetS DNA sequences were assembled and aligned using Vector NTI (InforMax, Bethesda, Md.).

Plasmids containing mutant metS genes were transformed into BL21 Star™ (DE3) cells for expression (Invitrogen, Carlsbad, Calif.). Expression was analyzed from 10 ml cultures of bacteria in F-media (1.4% yeast extract, 0.8% tryptone, 1.2% K₂HPO₄, and 0.12% KH₂PO₄, pH to 7.2). The culture was incubated at 37° C., and mixed by shaking at 200 rpm. Expression of mutant S. aureus MRS proteins was induced by addition of IPTG to 1 mM when the culture reached an OD₆₀₀ of 0.6-0.8. Total protein was extracted and analyzed by SDS-PAGE using 4-12% acrylamide gradient pre-cast gels (Novex NuPAGE-Invitrogen) using MOPS running buffer ((Invitrogen, Carlsbad, Calif.). A very intense protein band corresponding to S. aureus MRS migrated above the 70 kDa molecular weight standards of the 10 kDa protein ladder (Gibco, Rockville, Md.). This protein was observed as a distinct band in the induced cultures, but was not observed in the un-induced controls (FIG. 7).

For purification of mutant proteins, 2 L cultures in F-media were prepared as described above. The bacteria were harvested two hours post-induction by centrifugation and an equal amount (v/w) of 50 mM Tris (pH 7.5) and 10% sucrose solution was added to the cell paste. The resulting slurry was fast frozen in liquid nitrogen and stored at −80° C. until further use. Lysis is accomplished by creation of spheroplasts of the cells carrying the over-expressed S. aureus MRS proteins. First, a 1:1 suspension of frozen cells in Tris-sucrose which had been stored at −80° C. is added to tris-sucrose that is pre-warmed to 55° C. (2.75 ml/g of cells). To the stirred mixture, 0.5 M 1,4-dithiothreitol (DTT) (0.05 ml/g of cells) and lysis buffer (2M NaCl, 0.3M spermidine in Tris-sucrose adjusted to pH 7.5) (0.25 ml/g of cells) are added. The presence of 18 mM spermidine keeps the nucleoid condensed within partially disrupted cells and displaced DNA binding proteins. The pH of the slurry is adjusted to pH 8.0 by the addition of 2 M Tris base, and lysozyme is added re-suspended in 5.0 ml of Tris-sucrose buffer (5 mg lysozyme/g of cells). The slurry is distributed into 250 or 500 ml centrifuge bottles after stirring 5 min and incubated at 4° C. for 1 hour. The centrifuge bottles are then placed in a 37° C. swirling water bath and gently inverted every 30 seconds for 4 minutes. The supernatant is separated form insoluble cellular debris by centrifugation (23,000×g, 60 min, 4° C.). The recovered supernatant constitutes Fraction I (Fr I).

To Fr I, ammonium sulfate (0.226 g to each initial ml Fraction I-40% saturation) is added over a 15 min interval. The mixture is stirred for an additional 30 min at 4° C. and the precipitate is collected by centrifugation (23,000×g, 45 min, 0° C.). Additional ammonium sulfate (0.065 g to each initial ml FrI) is added to the supernatant resulting in 50% saturation. The mixture is stirred for an additional 30 min at 4° C. and the precipitate is collected by centrifugation (23,000×g, 45 min, 0° C.). The resulting pellets are quick frozen by immersion in liquid nitrogen and stored at −80° C.

Assays for inhibition of tRNA^{Met} aminoacylation are carried out as described in Example 2 with the exception that the reactions are not transferred out of the 96-well polypropylene plates (Costar). The reaction is terminated by the addition of 5 μl of 0.5 M EDTA to each well. To measure the activity, 150 μl of either Phosphodiesterase (PDE) Assay Beads or Polylysine Coated Yttrium Silicate SPA Beads (200 μg) (Amersham Biosciences) are added to the reaction. The plate is sealed with clear PVC seal and incubated at room temperature for 15 min. The plates are then centrifuged at 2000 rpm for 2 min in an Eppendorf Centrifuge 5810R to pellet the beads. Reaction products are counted using a TopCount-NXT microplate scintillation and luminescence counter (Packard BioScience).

Assays for inhibition of the ATP:PPi exchange reaction of the S. aureus MRS are basically as described in Bullard et al, 1999, J. Mol. Biol., 288, pp 567-577 and Bullard et al, 2000, Biochim. Biophys. Acta, 1490, pp 245-258. An exception is that IC50 is determined for inhibition of the reaction by Compound 1. Briefly, an enzyme mix (18 μl) (different amounts of S. aureus MRS, 45 mM Tris-HCl (pH 8.0), 275 μg/ml BSA, 1.8 mM DTT, 7.0 mM MgOAc) is mixed with 4.0 μl of serial dilutions of inhibitor in 96-well polypropylene plates (Costar). The substrate mix (20 μl) (71.0 mM Tris-HCl, (pH 8.0), 80.0 mM KCl, 4.5 mM MgOAc, 4.5 mM DTT, 4.5 mM ATP, 180 μg/ml BSA, 3.6 mM methionine, 3.6 mM [³²P]PPᵢ (50 cpm/pmol)) is added to the mix and the reaction is allowed to incubate at room temperature for 20 min. Reactions are stopped by spotting 2.0 μl aliquots of the reaction mix on PEI cellulose TLC plates (Sigma-Aldridge). ATP and PPᵢ are separated by TLC using 4M urea, 0.75 M KPᵢ (pH 3.5) as a mobile phase. ATP, PPᵢ, and Pᵢ are quantitated using a Storm 840 Phosphoimiager (Molecular Dynamics).

Assays for determining kinetic parameters (Kₘ, K_{cat}) are as described in Bullard et al, 1999, J. Mol. Biol., 288, pp 567-577 and Bullard et al, 2000, Biochim. Biophys. Acta, 1490, pp 245-258.

What is claimed is:

1. An isolated mutant S. aureus MRS polypeptide comprising an isolated polypeptide having at least one amino acid changed relative to SEQ ID NO: 3 or a fragment of SEQ ID NO:3, and wherein the mutant S. aureus MRS polypeptide is associated with decreased susceptibility to an antagonist of the polypeptide.

2. The method of claim 1, wherein the antagonist is N-(4-bromo-5-(1-fluorovinyl)-3-methylthiophen-2-ylmethyl)-N′-(1H-quinolin-4-one)propane-1,3-diamine.

3. The isolated S. aureus mutant MRS polypeptide of claim 1, wherein said isolated polypeptide comprises a mutation selected from the group consisting of L19F, T50A, E52D, G54S, G54A, I57N, I57T, Q58L, A61V, A61T, A64P, A64S, A77V, E98G, I94N, R100S, V108M, V108L, I238F, L213W, V215A, V215I, G223C, P230T, I238F, V242F, V242I, A247E, L257P, M269I, and V296F, and any combination of the foregoing.

4. The isolated S. aureus mutant MRS polypeptide of claim 1, wherein said isolated polypeptide exhibits at least one change in a biological function compared with a protein substantially corresponding to a wild type MRS protein.

5. The isolated S. aureus mutant MRS polypeptide of claim 1, wherein said change is selected from the group consisting of reduction of synthetase activity and increase in fitness burden.

6. A method, comprising
(a) contacting a compound to be tested with an isolated mutant MRS polypeptide; and
(b) determining whether the compound is a ligand of said polypeptide, wherein formation of a complex between the compound and the polypeptide is indicative that the compound is a ligand.

7. The method of claim 6, wherein the compound to be tested is selected from the group consisting of peptides, carbohydrates, vitamins, nucleic acids, botanically-derived compounds, organic compounds, pharmaceutical agents.

8. The method of claim 6, wherein the mutant bacterial MRS polypeptide is a mutant S. aureus MRS polypeptide.

9. The method of claim 8, wherein the mutant S. aureus MRS polypeptide is associated with decreased susceptibility to N-(4-bromo-5-(1-fluorovinyl)-3-methylthiophen-2-ylmethyl)-N′-(1H-quinolin-4-one)propane-1,3-diamine.

10. The method of claim 9, wherein the mutant S. aureus MRS polypeptide comprises a mutation selected from the group consisting of L19F, T50A, E52D, G54S, G54A, I57N, I57T, Q58L, A61V, A61T, A64P, A64S, A77V, E98G, I94N, R100S, V108M, V108L, I238F, L213W, V215A, V215I, G223C, P230T, I238F, V242F, V242I, A247E, L257P, M269I, and V296F, and any combination of the foregoing.

11. The method of claim 6, wherein the mutant bacterial MRS polypeptide is selected from the group consisting of a full length polypeptide and a fragment of a full length polypeptide.

12. The method of claim 6, wherein the formation of a complex is monitored by detecting or measuring a synthetase activity or cellular response by said mutant MRS protein in response to binding of a ligand thereto.

13. A method comprising
a) contacting compound to be tested with an isolated mutant MRS polypeptide with methionine under conditions suitable for binding of methionine to said polypeptide; and
b) detecting or measuring the formation of a complex between said polypeptide and methionine; and
c) determining whether the compound is a ligand said polypeptide, wherein inhibition of complex formation by the compound is indicative that the compound is a ligand of the polypeptide.

14. The method of claim 13, wherein said methionine is labeled with a detectable label.

15. The method of claim 13, wherein the inhibition of complex formation is monitored by detecting or measuring a decrease in synthetase activity or cellular response by said mutant MRS protein in response to binding of a ligand thereto.

16. The method of claim 13, wherein the compound to be tested is selected from the group consisting of peptides, carbohydrates, vitamins, nucleic acids, botanically-derived compounds, organic compounds, pharmaceutical agents.

17. The method of claim 13, wherein the mutant bacterial MRS polypeptide is a mutant S. aureus MRS polypeptide.

18. The method of claim 17, wherein the mutant S. aureus MRS polypeptide is associated with decreased susceptibility to N-(4-bromo-5-(1-fluorovinyl)-3-methylthiophen-2-ylmethyl)-N′-(1H-quinolin-4-one)propane-1,3-diamine.

19. The method of claim 18, wherein the mutant S. aureus MRS polypeptide comprises a mutation selected from the group consisting of L19F, T50A, E52D, G54S, G54A, I57N, I57T, Q58L, A61V, A61T, A64P, A64S, A77V, E98G, I94N, R100S, V108M, V108L, I238F, L213W, V215A, V215I, G223C, P230T, I238F, V242F, V242I, A247E, L257P, M269I, and V296F, and any combination of the foregoing.

20. The method of claim 13, wherein the mutant bacterial MRS polypeptide is selected from the group consisting of a full length polypeptide and a fragment of a full length polypeptide.
